(19) United States
(12) Patent Application Publication
(10) Pub. No.: US 2016/0250014 A1
(43) Pub. Date: Sep. 1, 2016

(54) SUPPORT ASSEMBLY FOR A REPLACEMENT LIGAMENT AND ASSOCIATED METHOD

(71) Applicants: Andrew Michael WILLIAMS, West Yorkshire (GB); XIROS LIMITED, Yorkshire (GB)

(72) Inventors: Andrew Michael Williams, Leeds (GB); Jonathan Lorrison, Leeds (GB); Bahaa Botros Seedhom, Leeds (GB); David John Beevers, Leeds (GB)

(21) Appl. No.: 15/032,948

(22) PCT Filed: Oct. 29, 2014
(86) PCT No.: PCT/GB2014/053206

§ 371 Date: Apr. 28, 2016

(30) Foreign Application Priority Data
Oct. 30, 2013 (GB) 1319168.9

(51) Int. Cl. A61F 2/08; A61B 17/04

(52) U.S. Cl. CPC A61F 2/0811; A61B 17/0401; A61F 2002/0823; A61F 2002/0852; A61F 2002/0882; A61B 2017/0404; A61B 2017/0414

(57) ABSTRACT

A support assembly for a replacement ligament is disclosed, for positioning the ligament in a bone tunnel formed in a joint between two adjacent bones and reinforcing the joint. The support assembly has a particular use in the reconstruction of the anterior cruciate ligament (ACL). One disclosed support assembly (24) comprises a fixation device (28) for a replacement ligament (26), the fixation device adapted to overlie a mouth (36) of a bone tunnel (16) and having a bone facing surface (34) which can abut an external surface (38) of a bone (12) to thereby position the ligament in the bone tunnel and support tensile loading on the ligament; a support element (30) for the replacement ligament, the support element being coupled to the fixation device and adapted to receive the ligament so that the ligament can be secured to the fixation device and thus positioned within the bone tunnel when the fixation device is located so that it overlies the tunnel mouth; and an extra-articular reinforcement element (32), the extra-articular reinforcement element being coupled to the fixation device and adapted to extend from the fixation device to an anchor point (54, 56) which is remote from the mouth of the bone tunnel, to provide extra-articular reinforcement for the joint.

The present invention relates to a support assembly for a replacement ligament, for positioning the ligament in a bone tunnel formed in a joint between two adjacent bones. In particular, but not exclusively, the present invention relates to a support assembly for use in the reconstruction of the anterior cruciate ligament (ACL). The present invention also relates to a method of locating a replacement ligament in a bone tunnel formed in a joint between two adjacent bones.

In the implantation of a replacement ligament in a bone joint, for example the knee joint between tibial and femoral components (bones), it is usual to drill tunnels through the bones, and to pull the replacement ligament through the tunnels until a required position is reached within the joint. The ligament is then anchored against linear movement in either direction.

The replacement ligament will typically be autogenous tissue harvested from the patient, or allogenic tissue harvested from a suitable donor, although xenograft tissue taken from an animal could conceivably be used. Suitable tissue in the example of ACL surgery includes hamstring tendon. Prosthetic ligaments can also be used, made of synthetic material, provided that it is of a suitable implantable nature, and which may be woven.

Prior techniques developed for ACL reconstruction involve the use of an elongate guide element which serves to secure one end of the replacement ligament against axial movement in one direction, and which can serve to guide the implantation of the ligament. The guide element is of a construction which is such that it does not need to be anchored in position by physical intrusion into the bone.

In one such prior technique, the guide element passes through the drilled-out bone tunnels and, upon exiting an upper mouth of one of the tunnels (for example when it projects upwardly out of the femoral component), a simple manipulation of the device causes it to overlie the mouth of the tunnel, abutting the cortical bone. The guide element thus provides tensile restraint for the replacement ligament attached to the guide element. Guide elements of this type are known in the industry as ‘Endobuttons™’.The guide element, or Endobutton™, is capable of being manipulated between a pulling position, in which it has a reduced lateral extent relative to the pulling direction, to an anchoring position, in which it has a maximum lateral extent relative to the pulling direction (for overlying the mouth of the bone tunnel).

In a variation on this technique, the replacement ligament may be inserted in a reverse direction (from the femoral component into the tibial component), trailing an assembly including a guide element which can overlie the mouth of a bone tunnel in the fashion described above, to provide tensile restraint for the ligament. As the guide element trails the replacement ligament, it is not necessary for it to pass along the bone tunnels. This means that the guide element does not need to be sized for passage along the tunnel and subsequent flipping. Accordingly, guide elements of larger dimension may be employed, which can overlie the tunnel mouth around its entire perimeter. This provides certain advantages. In particular, the risk of torsional loading on the guide element rotating it to a position where it could be flipped and drawn back into the bone tunnel can be avoided.

When performing an ACL reconstruction technique, a surgeon must make a careful assessment of the suitable location and direction of the bone tunnels which are to be formed, in particular that which is to be formed in the femoral component of the knee joint. Typically, the surgical techniques which have been developed involve the drilling of a femoral bone tunnel which has an interior opening in the lateral condyle that is displaced from the ‘isometric’ position where the natural ACL was previously attached to the bone. The primary reason for this was a desire to increase rotational stability of the joint. However, one significant drawback to this is a resultant increase in loading on the replacement ligament during flexure and extension of the joint, which is undesirable.

Another prior technique is disclosed in International Patent Publication No. WO-89/10101, and employs a prosthetic ligament comprising pockets which receive bone plugs, for locating the ligament within the knee joint. The bone plugs are positioned in enlarged diameter portions of bone tunnels in the femoral and tibial components, and act to both anchor and support tensile loading on the ligament. Modifications to the technique disclosed in WO-89/10101 involve the location of a prosthetic ligament in the bone tunnels which has an extra-articular reinforcement portion. The extra-articular reinforcement portion extends out of the bone tunnel in the femoral component, and can be wrapped around the femoral component and anchored to the tibial component. Typically, a second bone tunnel is drilled in the tibial component, and the extra-articular portion extends through and is anchored in the second tibial tunnel.

There is a desire to improve upon the above prior techniques, in particular those employing ligaments with extra-articular reinforcement portions. There is also a desire to facilitate the use of extra-articular reinforcement portions in other ACL reconstruction techniques.

According to a first aspect of the present invention, there is provided a support assembly for positioning a replacement ligament in a bone tunnel formed in a joint between two adjacent bones and reinforcing the joint, the support assembly comprising:

a fixation device for the replacement ligament, the fixation device adapted to overlie a mouth of the bone tunnel and having a bone facing surface which can abut an external surface of the bone to thereby position the ligament in the bone tunnel and support tensile loading on the ligament;

a support element for the replacement ligament, the support element being coupled to the fixation device and adapted to receive the ligament so that the ligament can be secured to the fixation device and thus positioned within the bone tunnel when the fixation device is located so that it overlies the tunnel mouth; and

an extra-articular reinforcement element, the extra-articular reinforcement element being coupled to the fixation device and adapted to extend from the fixation device to an anchor point which is remote from the mouth of the bone tunnel, to provide extra-articular reinforcement for the joint.

Advantageously, the support assembly of the present invention may provide the ability to position a replacement ligament within a bone tunnel with a degree of adjustability, to support tensile loading on the ligament, and to reinforce the joint by means of the extra-articular reinforcement element. In particular, the provision of an assembly comprising such a fixation device may offer advantages in terms of the ability to adjust the assembly to suit patients of different sizes (and thus different bone dimensions), whilst also providing for reinforcement of the joint using the extra-articular reinforcement element.

The support element may take the form of, or may define, a loop. The loop may define an eye through which the ligament can pass so that the ligament can be positioned in the bone tunnel. The loop may be of a flexible material and may extend through a pair of apertures in the fixation device. The loop may be an endless loop. The loop may be formed by knotting or stitching. The loop may be a rigid loop secured to or formed as part of the fixation device.

The reinforcement element may be coupled to the fixation device by the support element. Accordingly, the support element may serve for coupling both the ligament and the reinforcement element to the fixation device.

The support element for the ligament may be a first support element, and the assembly may comprise a second support element which may be for the reinforcement element, for coupling the reinforcement element to the fixation device. The first and second support elements may each have the further features of the support element defined above. The first and second support elements may extend through a common pair of apertures in the fixation device. The fixation device may comprise a pair of apertures for the first support element, and a separate pair of apertures for the second support element.

The reinforcement element may be directly coupled to the fixation device, and may be coupled by passing the reinforcement element through at least one aperture of the fixation device.

The reinforcement element may form a pulling element for pulling the support assembly and a trailing ligament coupled to the fixation device along the bone tunnel.

The fixation device may serve for guiding the ligament along the bone tunnel, and may be a generally elongate device capable of being manipulated between a pulling position, in which it has a reduced lateral extent relative to a pulling direction, to an anchoring position, in which it has a maximum lateral extent relative to the pulling direction. This may facilitate passage of the device along the bone tunnel and subsequent manipulation of the device to the position where it overlies the mouth of the tunnel. The reinforcement element may form a manipulating element for manipulating the fixation device from the pulling position to the anchoring position.

The fixation device may comprise a pair of spaced apertures for the reinforcement element, the reinforcement element passing through the pair of apertures so that it is secured to the fixation device. This may provide improved torsional stability of the fixation device under load.

The reinforcement element may also form or define the support element. The reinforcement element may pass through a first reinforcing aperture in the fixation device in a first direction and then through a second reinforcing aperture (which may be spaced from the first aperture) in a second direction, to thereby form a loop defining an eye which can receive the ligament.

The reinforcement element may be an elongate element of a suitable implantable material, and may be woven. The reinforcement element may be a woven elongate tape. The reinforcement element, when coupled to the fixation device, may be folded so that it comprises first and second legs. The reinforcement element may be coupled to the fixation device by securing the first and second legs together. The legs may be secured together via a knot or by stitching. Other securing methods may be employed. The reinforcement element may be at least partly tubular, at least one aperture provided in a wall of one of the first and second legs so that the other one of the first and second legs may pass into the aperture and along the inside of the other leg.

The reinforcement element may comprise a coupling region part way along a length of the element between opposed first and second ends of the element, the coupling region adapted for coupling the reinforcement element to the fixation device. The coupling region may have at least one dimension which is less than a corresponding dimension of a remainder or majority of the element. The at least one dimension may be a width.

The reinforcement element may be coupled to the fixation device in such a way that the reinforcement element defines a plurality of loops which together form the support element. Advantageously, the formation of a plurality of loops may facilitate the provision of a coupling region having the reduced dimension discussed above (which may in turn facilitate coupling of the reinforcement element to the fixation device), whilst providing a support element of sufficient strength to support tensile loading on the ligament during use.

The reinforcement element may be coupled to the fixation device in such a way that the length of the or each loop is adjustable. This may be advantageous in that it may facilitate adjustment of the assembly to suit patients of different sizes. In particular, it may enable the accommodation of patients having different bone sizes and, where the replacement ligament is natural tissue (such as a hamstring tendon), may enable adjustment to suit the particular length of the harvested tissue.

The support assembly may comprise a guide member for the reinforcement element, the guide member being implantable in a bone of the joint and shaped to cooperate with the reinforcement element to provide control of a direction of loading applied to the fixation device by the reinforcement element during use. The guide member may be a post, pin or the like and may define a surface around which the reinforcement element can pass so that the reinforcement element extends from the fixation device, around the guide pin and then to the anchor point. In use, the reinforcement element may extend from the fixation device to the guide member in a first direction, and then from the guide member to the anchor point in a second, different direction (which may be non-parallel to the first direction). Alternative guide members may be employed, such as a staple. Typically the staple would clamp the reinforcement element to the bone surface, but in a variation the staple may not clamp the reinforcement element, or may carry a post or the like around which the reinforcement element may pass.

According to a second aspect of the present invention, there is provided a method of locating a replacement ligament in a bone tunnel formed in a joint between two adjacent bones and of reinforcing the joint, the method comprising the steps of:

securing a replacement ligament to a fixation device of a support assembly using a support element coupled to the fixation device;

inserting the ligament into the bone tunnel;

locating the fixation device so that it overlies a mouth of the bone tunnel with a bone facing surface of the fixation device abutting an external surface of the bone, to thereby position the ligament secured to the fixation device within the bone tunnel and to support tensile loading on the ligament;

extending an extra-articular reinforcement element coupled to the fixation device from the fixation device to an anchor point which is remote from the mouth of the bone tunnel; and

anchoring the reinforcement element at the anchor point, to provide extra-articular reinforcement for the joint.

The method may comprise the further steps of: implanting a guide member for the reinforcement element in a bone of the joint; and arranging the reinforcement element so that it cooperates with the guide member, so that the guide member provides control of a direction of loading applied to the fixation device by the reinforcement element during use. The step of arranging the reinforcement element so that it cooperates with the guide member may comprise passing the reinforcement element around a surface of the guide member. The method may comprise locating the reinforcement element so that it extends from the fixation device, around the guide pin and then to the anchor point. The method may comprise locating the reinforcement element so that it extends from the fixation device to the guide member in a first direction, and then from the guide member to the anchor point in a second, different direction (which may be non-parallel to the first direction).

Further features of the method of the second aspect of the invention may be derived from the text set out above relating to the assembly of the first aspect of the invention.

Embodiments of the present invention will now be described, by way of example only, with reference to the accompanying drawings, in which:

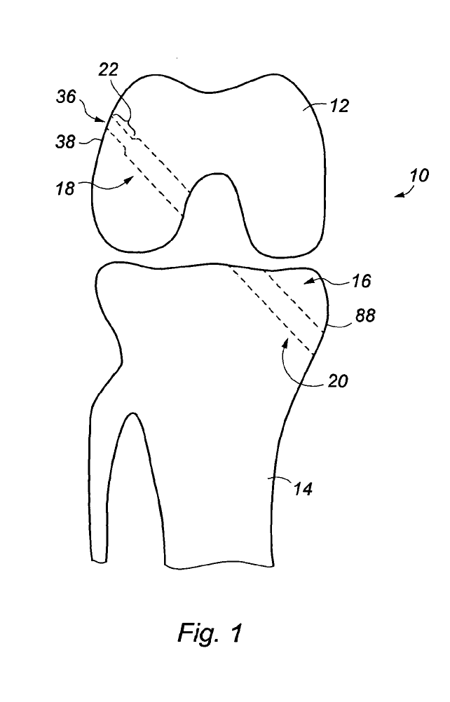

FIG. 1 is a schematic front view of a joint between two adjacent bones which, in this case is a knee joint between a femoral component and a tibial component;

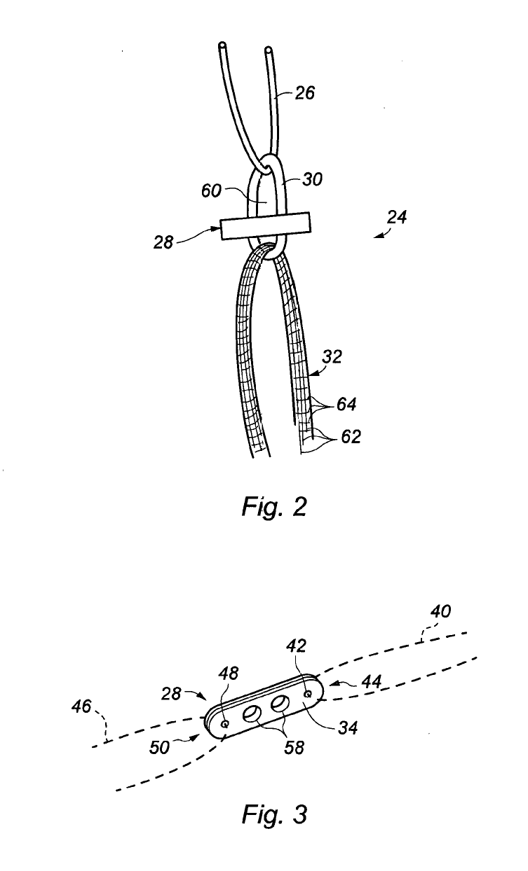

FIG. 2 is a view of a support assembly for a replacement ligament in accordance with an embodiment of the present invention;

FIG. 3 is a perspective view of a fixation device of the support assembly shown in FIG. 1;

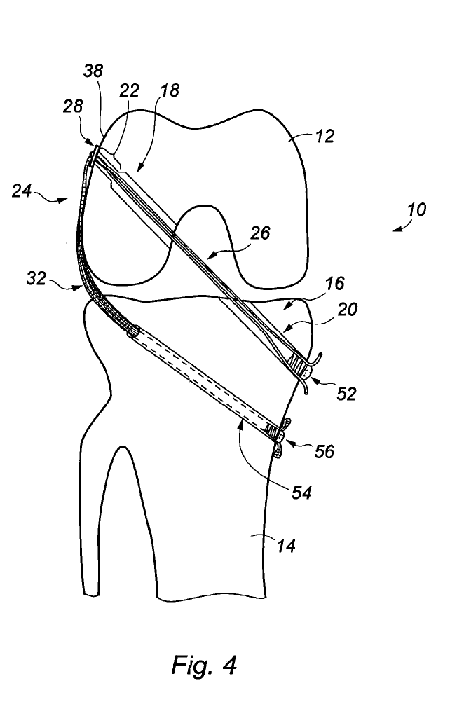

FIG. 4 is a view similar to FIG. 1 but showing the support assembly of FIG. 2 following implantation, with a replacement ligament coupled to the support assembly located in a bone tunnel formed in the bones of the joint;

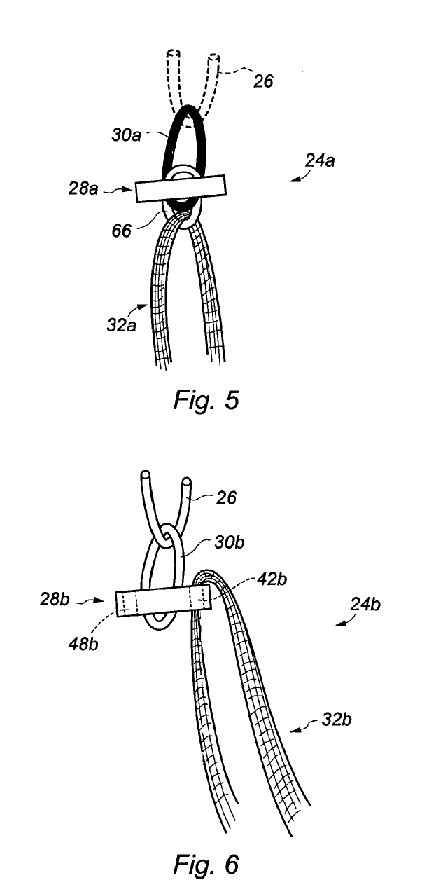

FIGS. 5 to 8 are views of support assemblies for replacement ligaments in accordance with further embodiments of the present invention;

FIG. 9 is a view of the support assembly of FIG. 8, showing a fixation device of the assembly from the side, rather than in perspective view as in FIG. 8;

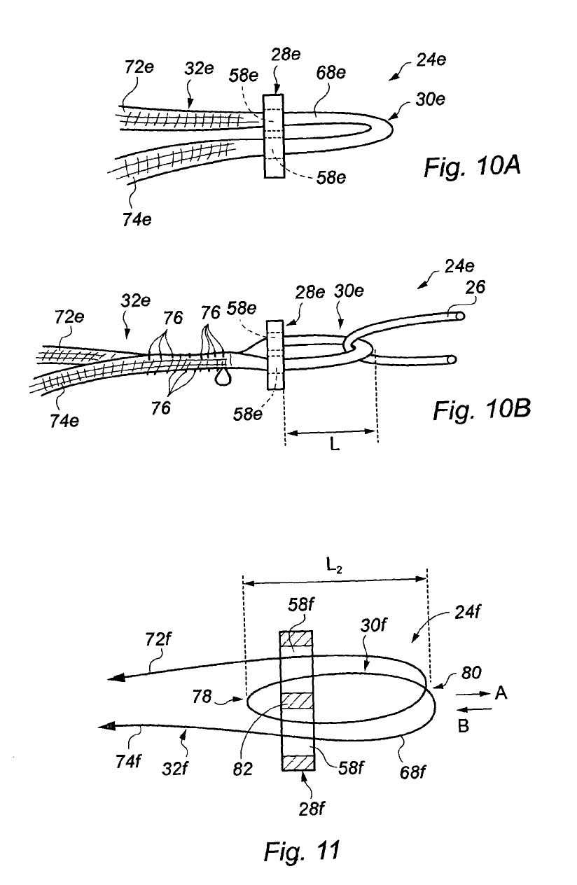

FIGS. 10A and 10B show a support assembly for a replacement ligament in accordance with another embodiment of the present invention during different stages of construction of the assembly and/or a technique to implant a ligament using the assembly;

FIGS. 11 to 14 are views of support assemblies for a replacement ligament in accordance with further embodiments of the present invention;

FIGS. 15A, B and C are views of support assemblies for replacement ligaments in accordance with further embodiments of the present invention, each of which comprises a fixation device of a different type to that shown in the embodiments of FIGS. 2 to 14;

FIG. 16 is a view of a support assembly for a replacement ligament in accordance with another embodiment of the present invention, comprising a fixation device of the type shown in FIGS. 15A to C; and

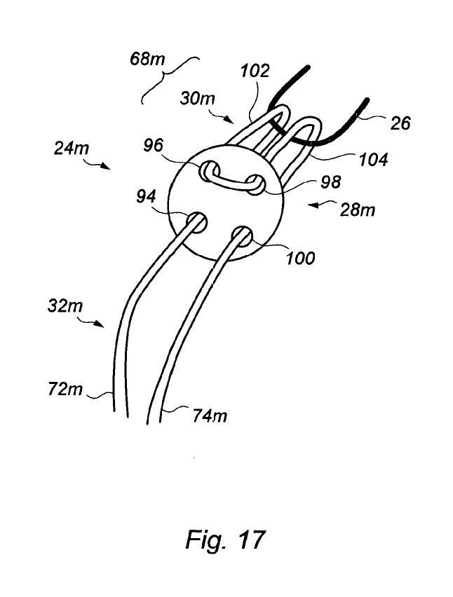

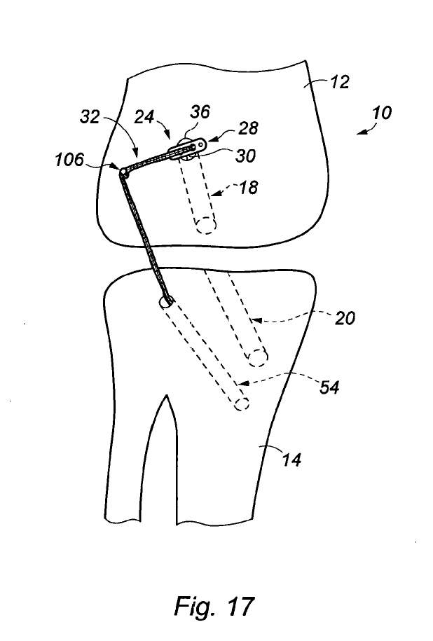

FIG. 17 is a side (or lateral) view of the bone joint shown in FIG. 1, illustrating an optional further feature of any one of the support assemblies shown and described in FIGS. 2 to 16.

Turning firstly to FIG. 1, there is shown a schematic front view of a joint 10 between two adjacent bones 12 and 14, which in this case is a knee joint between a femoral component 12 of the joint and a tibial component 14. The knee joint 10 is shown following preparation for the implantation of a replacement ligament in an ACL reconstruction technique. This has involved the drilling of a bone tunnel 16, indicated in broken outline, and which comprises a femoral tunnel portion 18 and a tibial tunnel portion 20. The femoral tunnel portion 18 may be a stepped tunnel having a smaller diameter section 22, the purpose of which will be understood by persons skilled in the art, but which will be briefly discussed below.

Turning now to FIG. 2, there is shown a support assembly for a replacement ligament in accordance with an embodiment of the present invention, the assembly indicated generally by reference numeral 24. The assembly 24 is for positioning a replacement ligament 26 in the bone tunnel 16 formed in the knee joint 10 between the femoral and tibial components 12 and 14, and also serves for reinforcing the joint. Typically, the replacement ligament 16 will be autogenous or allogenic tissue, and may be a hamstring tendon. However, prosthetic ligaments of synthetic material can be employed, provided that they are of a suitable implantable nature. Xenograft tissue could conceivably also be employed.

The assembly 24 generally comprises a fixation device 28 for the ligament 26, for positioning the ligament in the bone tunnel 16 and supporting tensile loading on the ligament; a support element 30 for the ligament 26, which is coupled to the fixation device 28 and adapted to receive the ligament; and an extra-articular reinforcement element 32. The fixation device 28 is shown in more detail in the perspective view of FIG. 3, and is an external fixation device. The device 28 is adapted to overlie a mouth 36 of the bone tunnel 16, in this case a mouth of the femoral tunnel portion 18. The device 28 comprises a bone facing surface 34 which can abut an external surface 38 of the femoral component 12, to thereby position the ligament 26 secured to the fixation device 28 in the bone tunnel 16, and to support tensile loading on the ligament by contact between the bone facing surface 34 and the external bone surface 38. The ligament 26 is thus positioned without requiring that the fixation device 28 intrude into the bone of the femoral component 12. The ligament 26 is secured to the fixation device 28 via the support element 30 so that, when the fixation device 28 is located overlying the tunnel mouth 36, the ligament 26 is positioned within the bone tunnel and the tensile load supported as discussed above. The reinforcement element 32 is also coupled to the fixation device 28, and extends from the device to an anchor point which will be shown and discussed below. The anchor point is remote from the mouth 36 of the bone tunnel 16, so that the reinforcement element 32 provides extra-articular reinforcement for the knee joint 10.

FIG. 4 is a view similar to FIG. 1, but showing the support assembly 24 of FIG. 2 following implantation, with the replacement ligament 26 located in the bone tunnel 16. For ease of illustration and understanding, the bone tunnel 16, assembly 24 and ligament 26 are mainly shown in solid outline. The fixation device 28 is generally elongate, and acts as a guide for the ligament 26 for passage of the ligament along the bone tunnel 16. In the illustrated embodiment, the fixation device 28 takes the general form of an Endobutton™.The assembly 24 and ligament 26 are drawn up through the tibial tunnel portion 20 and through the femoral tunnel portion 18 using a pulling suture 40, shown in broken outline in FIG. 3. The pulling suture 40 extends through a pulling aperture 42 in the fixation device 28, which is provided towards an end 44 of the device, to cause it to adopt a pulling position in which it has a reduced lateral extent relative to the pulling direction.

On emergence of the fixation device 24 from the mouth 36 of the femoral tunnel portion 18, the fixation device 28 can be “flipped” and so moved to an anchoring position, shown in FIG. 4, in which it has a maximum lateral extent relative to the pulling direction. The bone facing surface 34 of the fixation device 28 is then supported against the external surface 38 of the bone. The fixation device 28 is flipped using a flipping suture 46, also shown in broken outline in FIG. 3, which extends through a flipping aperture 48 provided in a second end 50 of the fixation device. The femoral tunnel portion 18 is stepped, as discussed above, including the smaller diameter section 22. The purpose of this is to provide a mouth 36 which is of smaller diameter than a main part of the tunnel portion 18, to reduce the risk of the fixation device 28 being drawn back into the tunnel, whilst also facilitating passage of the device along the main (larger diameter) part of the tunnel portion. Following positioning of the fixation device 28 against the surface of the femoral component 12, the ligament 26 located in the bone tunnel 16 is tensioned and anchored in position at the opposite end of the bone tunnel 16, using a suitable anchoring element such as a screw 52.

In the embodiment of FIG. 2, the support element 30 also serves for coupling the reinforcement element 32 to the fixation device 28. Following positioning of the ligament 26 in the bone tunnel 16, and anchoring of the ligament, the reinforcement element 32 can be tensioned and similarly anchored. One typical method by which the reinforcement element 32 may be anchored is shown in FIG. 4, and involves forming a second tunnel 54 in the tibial component 14, passing the reinforcement element 32 along the second tunnel 54 and securing it using an anchoring element such as a screw 56. The anchor point for the reinforcement element 32 is thus remote from the mouth 36 of the bone tunnel 16, the anchor point effectively being the position of the anchor screw 56. In a variation on the disclosed technique, the reinforcement element may be anchored to an external surface of the tibial component 14, such as by a staple. Securement to the tibial component 14 is preferred, in order to provide appropriate reinforcement. However, in techniques involving location of the fixation device 28 adjacent a surface of the tibial component 14 (which will be discussed below), the reinforcement element 32 may be secured to the femoral component 12.

In addition to the pulling and flipping apertures 42 and 48, the fixation device 28 includes a pair of larger diameter apertures 58 which receive the support element 30, the apertures 58 disposed between the pulling and flipping apertures, in a direction along a length of the fixation device 28. The support element 30 is formed into a loop, and may be an endless loop manufactured and coupled to the fixation device 28 following the method disclosed in International patent publication number WO-99/47079, the disclosure of which is incorporated herein by way of reference. In the illustrated embodiment, the endless loop 30 defines an eye 60 which receives both the tendon 26 and the reinforcement element 32, on opposite sides of the fixation device 28, as best shown in FIG. 2. The reinforcement element 32 is elongate and of a woven synthetic implantable material comprising warps 62 and wefts 64, typically taking the form of a tape. Coupling of the reinforcement element 32 to the fixation device 28 by passing it through the eye 60 of the endless loop 30 provides the advantage that the loading imparted on the fixation device 28 by the reinforcement element 32 during use is applied to a generally central region of the fixation device 28. Depending upon the particular rotational orientation of the fixation device 24, and the direction of applied loading, this can assist in reducing or avoiding torsional forces in the fixation device 28, which might otherwise result in the device being rotated to a position where it could pass back into the mouth 36 of the femoral tunnel portion 18.

The support assembly 24 of the present invention provides numerous advantages over prior assemblies and associated techniques. In particular, the assembly enables positioning of the ligament 26 within the bone tunnel 16 and the support of tensile loading on the ligament whilst also providing an extra-articular reinforcement for the knee joint 10. Coupling of the ligament 26 to the fixation device 24 via the support element 30, and coupling of the reinforcement element 32 to the fixation device 24, facilitates this whilst also providing for good adjustability of the assembly and so potentially the position of the ligament 26.

Turning now to FIGS. 5 to 16, there are shown various support assemblies in accordance with further embodiments of the present invention, the assemblies indicated generally by reference numeral 24 together with a respective suffix “a” to “m”. Like components of the assemblies 24a to 24m with the assembly 24 of FIG. 2 share the same reference numerals with the addition of the respective suffix. Only the substantial differences between the assemblies 24a to 24m and the assembly 24 of FIG. 2 will be described in detail.

Accordingly and turning now to FIG. 5, there is shown a support assembly in accordance with another embodiment of the present invention, the assembly indicated generally by reference numeral 24a. In this embodiment, the support assembly 24a comprises a first support element 30a for ligament 26 (shown in broken outline), and a second support element 66 for an extra-articular reinforcement element 32a. The first and second support elements 30a and 66 both take the form of endless loops, of the type described above. Typically, the two loops 30a and 66 will be located in a common pair of apertures, similar to the apertures 58 shown in FIG. 3, which may then require to be of larger dimension. However, if desired, dedicated pairs of apertures (not shown) may be provided for each loop 30a and 66. The assembly 24a may offer the advantage that tensile loading in the ligament 26 is not transferred directly to the reinforcement element 32, and vice-versa.

Turning now to FIG. 6, there is shown a support assembly in accordance with another embodiment of the present invention, the assembly indicated generally by reference numeral 24b. In this embodiment, an extra-articular reinforcement element 32b is coupled directly to a fixation device 28b, passing through a pulling aperture 48. In this embodiment, the reinforcement element 32 thus additionally serves as a pulling element for drawing the fixation device 28b, a support element in the form of an endless loop 30b, and the trailing ligament 26 along the bone tunnel 16. A flipping suture (not shown) would be employed to flip the fixation device 28b, passing through a flipping aperture 48b. In a variation on the illustrated embodiment, the reinforcement element 32b may pass through the flipping aperture 48b, and be employed to flip the fixation device 28b. In this case, a pulling suture would pass through the pulling aperture 42b, and be employed to draw the assembly 24b through the bone tunnel 16.

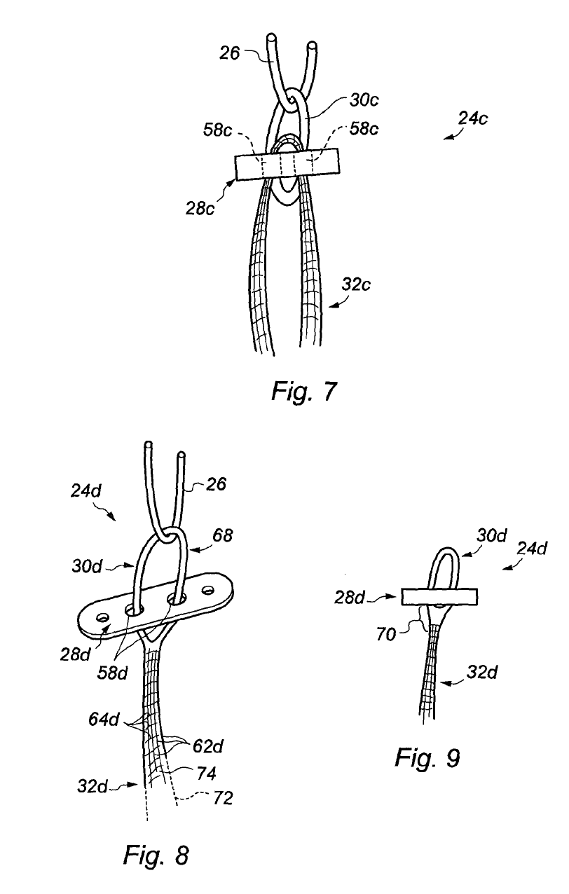

Turning now to FIG. 7, there is shown a support assembly in accordance with another embodiment of the present invention, the assembly indicated generally by reference numeral 24c. In this embodiment, a reinforcement element 32c is once again directly coupled to a fixation device 28c, in this case passing through a central pair of apertures 58c. The apertures 58c also receive a support element in the form of an endless loop 30c, by which ligament 26 is coupled to the fixation device 28c. However, separate apertures may be provided for the loop 30c and the reinforcement element 32 if desired. The embodiment of FIG. 7 offers the combined advantages that direct coupling of the reinforcement element 32c to the fixation device 28c avoids a direct transfer of loading from the ligament 26 to the reinforcement element 32c, whilst generally central location of the reinforcement element on the fixation device helps to avoid torsional loading on the fixation device.

Turning now to FIG. 8, there is shown a support assembly in accordance with another embodiment of the present invention, the support assembly indicated generally by reference numeral 24d. FIG. 9 is a view similar to FIG. 8, but showing a fixation device 28d from the side, rather than in perspective view as in FIG. 8. In this embodiment, a reinforcement element 32d also defines a support element for ligament 26. This is achieved by directly coupling the reinforcement element 32d to a fixation device 28d, the reinforcement element passing through a pair of apertures 58d in the fixation device to form the support element 30d, on the other side of the fixation device 28d.

To facilitate coupling of the reinforcement element 32d to the fixation device 28d, and so formation of the support element 30d, a dedicated coupling region 68 is formed which is part way along a length of the reinforcement element 32d between first and second ends of the element. The reinforcement element 32d is again an elongate woven tape comprising warps 62d and wefts 64d, and the coupling region 68 is free from wefts 64d, so that the coupling region is effectively a “weftless” region. This facilitates coupling of the reinforcement element 32d to the fixation device 28d, in that the reinforcement element 32d is less bulky in the weftless coupling region 68, with the result that the apertures 58d can be smaller than would otherwise be the case in order to accommodate the reinforcement element.

Typically, whipping would be applied to an area 70 of the reinforcement element 32d, to form the coupling region 68 into a loop, and to provide sufficient strength in the area 70 to prevent separation of legs 72 and 74 of the reinforcement element 32d. However, if desired, the legs 72 and 74 may additionally or alternatively be secured to one another, for example by stitching the legs together along their length or part of a length thereof. Typically, the position of the whipping in the area 70, and so a length of the loop of the support element 30d which is formed, will be pre-selected, so that the assembly is provided with the reinforcement element 32d pre-coupled to the fixation device 28d and secured by the whipping.

FIGS. 10A and 10B show a support assembly in accordance with another embodiment of the present invention, the assembly indicated generally by reference numeral 24e. The assembly 24e is in fact very similar to the assembly 24d of FIGS. 8 and 9, differing from the assembly 24d in that a reinforcement element 32e having a weftless coupling region 68e is provided which is not pre-secured as in the embodiment of FIGS. 8 and 9. A length of a loop formed by the coupling region 68e, forming the support element 30e, can therefore selected by a surgeon, and so finalised as part of the surgical procedure.

Specifically and as shown in FIG. 10A, the support assembly 24e is typically supplied with the reinforcement element 32e coupled to a fixation device 28e, with the weftless coupling region 68a passing through apertures 58e (shown in broken outline). A length L (FIG. 10B) of the loop, and so of the support element 30e thus formed, is selected depending upon factors including the length of the tendon 26, and dimensions of the femoral and tibial components 12, 14 and of the bone tunnel 16. The length L is adjusted simply by pulling on legs 72e and 74e of the reinforcement element 32e, or by pulling on the coupling region 68e. The legs 72e and 74e are then secured to one another by knotting or applying stitching ties, indicated schematically at 76 in the drawings. Alternatively, the assembly 24e may be supplied ready assembled, as shown in FIG. 10B, in a number of different sizes where each size has a different loop length L from which the optimum can be chosen and used depending on factors including dimensions of the femoral and tibial components 12 and 14, of the bone tunnel 16, and/or the ligament 26.

Turning now to FIG. 11, there is shown a support assembly in accordance with another embodiment of the present invention, the assembly indicated generally by reference numeral 24f. In this embodiment, the assembly 24f comprises a reinforcement element 32f which is shown in highly schematic form, but which again will typically take the form of an elongate woven tape. The reinforcement element 32f is coupled to a fixation device 28f, and defines a support element 30f in the form of an adjustable length loop. The loop 30f is formed by passing the reinforcement element 32f through a first aperture 58f in the fixation device 28f and then back around through a second aperture 58f, to form a first portion of the support loop 30f. The reinforcement element 32f is then passed back through the first aperture 58f and again through the second aperture, forming a second portion of the support loop 30f.

The loop 30f is formed by a coupling region 68f, which again may be a de-welted section of the reinforcement element 32f. A length L₂ of the loop 32f is adjustable as follows. The loop 30f has opposed ends 78 and 80. In use, the end 78 contacts a bridge 82 of the fixation device 28f, defined between the apertures 58f. Legs 72f and 74f of the reinforcement element 32f are initially not connected, and the length L₂ can be increased by pulling on the loop 30f in the area of the second end 80. This translates the legs 72f and 74f through the apertures 58f in the direction of the arrow A, to extend the loop 30f. Pulling the legs 72f and 74f back through the apertures 58f in an opposite direction B will shorten the length L₂ of the loop 30f. The legs 72f and 74f can then be secured together, for example by knotting or stitching, as described in relation to FIGS. 8 to 10B.

The overlapping of the reinforcement element 32f in the region of the second end 80 provides increased support for the ligament 26, when compared for example to the embodiments of FIGS. 8 to 10B. In particular, this may balance the reduction of material in the de-wefted coupling region 68f, compared to a remainder or main part of the reinforcement element 32f, in terms of supporting the loading imparted on the support loop 30f by the ligament. Furthermore, the additional friction which results from passage of the reinforcement element 32f around and through the fixation device 28f as described above may facilitate selection of a length L₂ of the loop 30f. This is because a larger force will be required to adjust the loop length, with the result that accidental variations in the length will be less likely to occur.

In a further variation on the embodiment of FIG. 11, the reinforcement element 32f may pass back through the apertures 58f through one or more further turns, to define a plurality of loops. Providing a plurality of loops may enable a width of the reinforcement element 32f to be further reduced in the coupling region 68f, compared to a remainder or main part of the reinforcement element, and/or may further balance the reduction of material. Also, whilst the reinforcement element 32f is wound through a single pair of apertures 58f, in a further variation, there may be more than two apertures so that the element 32f may only pass once through each aperture.

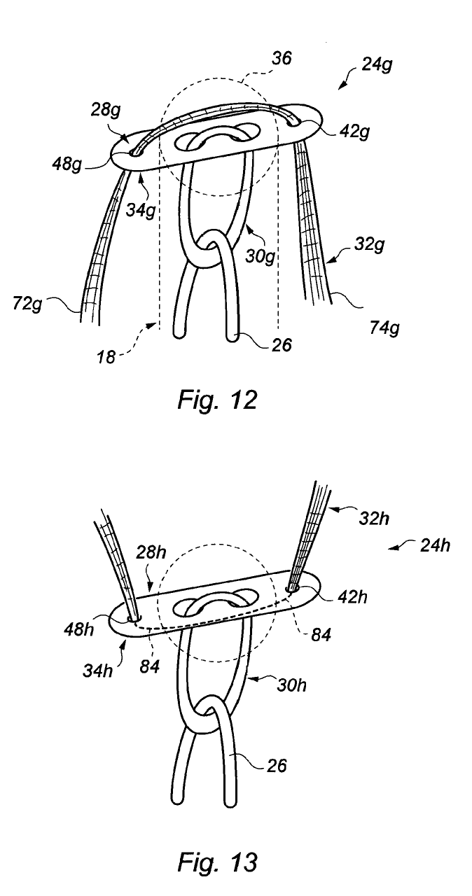

Turning now to FIG. 12, there is shown a support assembly in accordance with another embodiment of the present invention, the assembly indicated generally by reference numeral 24g. The assembly 24g is in fact of very like construction to the assembly 24c shown in FIG. 7, save that a reinforcement element 32g passes through both pulling and flipping apertures 42g and 48g. Typically, separate pulling and flipping sutures (not shown) will be provided for guiding the assembly 24g along the bone tunnel 16 and positioning a fixation device 28d of the assembly. The approximate location of the fixation device 28g, relative to the mouth 36 of the femoral tunnel portion 18, is shown in broken outline. It will be understood that, in use, legs 72g and 74g of the reinforcement element 32g must pass back away from the femoral bone surface 38, with the result that they will effectively be sandwiched between a bone facing surface 34g of the fixation device 28g and the bone surface 38.

FIG. 13 shows a support assembly in accordance with another embodiment of the present invention, the assembly indicated generally by reference numeral 24h. The assembly 24h is effectively the same as that shown in FIG. 12, except that a reinforcement element 32h passes through pulling and flipping apertures 42h and 48h of a fixation device 28h in the opposition direction to FIG. 12. Small portions 84 of the reinforcement element 32h, indicated in broken outline, are thus effectively sandwiched between a bone facing surface 34h and the femoral bone surface 38.

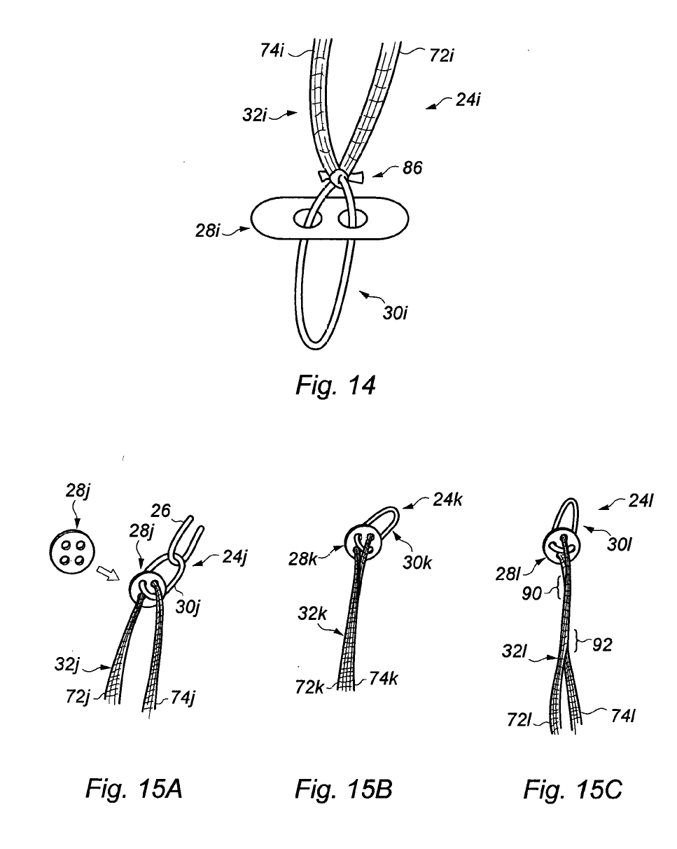

Turning now to FIG. 14, there is shown a support assembly in accordance with another embodiment of the present invention, the assembly indicated generally by reference numeral 24i. The assembly 24i is essentially the same as that shown in FIG. 8, with the exception that legs 72i and 74i of a reinforcement element 32i (which defines a support element 30i for a ligament) are secured together by a knot 86. The assembly 24i may be supplied with the reinforcement element 32i already secured to a fixation device 28i, and thus with the knot 86 between the legs 72i and 74i already formed. However, it may be desirable to supply the assembly 24i without the knot 86 having been formed, in a similar fashion to the assembly 24e in FIG. 10A, so that a surgeon may position the knot 86 appropriately to form a loop of a desired length, for supporting the ligament.

Whilst FIG. 1 illustrates a surgical technique in which fixation devices of the disclosed support assemblies are secured against an external surface 38 of a femoral component 12 of a knee joint 10, it will be understood by person skilled in the art that the fixation devices may be secured relative to an external surface of the tibial component 14. Furthermore, whilst the preceding embodiments are intended for insertion along the bone tunnel 16 from the tibial component 14 into the femoral component 12, it will be understood that many surgical techniques employ a reverse insertion procedure, passing from the femoral component into the tibial component.

FIGS. 15A, B and C illustrate support assemblies 24j, 24k and 24l which include respective fixation devices 28j, 28k and 28l intended for anchorage against an external surface 88 of the tibial component 14, and/or for insertion in the reverse direction. In each case, the support assemblies 24j, k and l are provided as trailing assemblies to the ligament 26. The ligament 26 is this inserted into and translated along the bone tunnel 16 trailing the assemblies 24j, k and l, such as by a suitable pulling suture secured to the ligament. The advantage of this is that the fixation devices 28j, k and l can be of larger dimension than would otherwise be the case, as they do not need to transit along the bone tunnel 16 and to then be flipped to an anchoring position, as is the case with the Endobutton type fixation devices shown 28 to 28i shown and described in FIGS. 2 to 14. One advantage of this is that the fixation devices 28j, k and l, which typically take the form of or define buttons, can all be dimensioned so that they overlie the mouth of the respective tunnel portion, reducing the possibility of the buttons being dragged back into the bone tunnel in the event of torsional loading on the buttons causing them to twist.

The assemblies 24j, k and l include respective reinforcement elements 32j, k and l as well as support elements for ligament 26 which take the form of endless loops 30j, k and l of the type described above. It will be understood however that variations on the disclosed embodiments may include the features of any one of the assemblies shown and described in FIGS. 2 to 14. For example, the reinforcement elements 32j, k or l may define the support elements 30j, k or l.

The reinforcement element 32j of the assembly 24j comprises legs 72j and 74j which can be secured by forming a knot. The reinforcement element 32k of the assembly 24k includes legs 72k and 74k which are secured together by stitching. The reinforcement element 32l of the assembly 24l includes legs 72l and 74l. The reinforcement element 32l is tubular along at least part, and optionally all of its length. Openings are formed in a wall of the leg 72l in regions 90 and 92, and the leg 74l is inserted through the opening 90, passing along the inside of the tubular leg 72l to opening 92, where it exits, as shown in the drawing. The legs 72l and 74l may be secured by stitching, for example in the regions 90 and 92. The reinforcement elements 32j, k and/or l may all be elongate woven tapes, and may comprise de-welted portions by which the elements are coupled to the respective fixation devices 28j, k, l.

FIG. 16 illustrates a support assembly 24m in accordance with another embodiment of the present invention, similar to those shown in FIGS. 15A to C, but in which a reinforcement element 32m also forms a support element 30m for ligament 26 (in a similar fashion to the assembly 24d of FIG. 8). A fixation device 28m in the form of a button includes apertures 94, 96, 98 and 100. Again, the reinforcement element 32m may be de-wefted in a coupling region 68m which defines the support element 30m. The reinforcement element 32m has legs 72m and 74m, and is fed through the aperture 94 and then back through the aperture 96 so as to form a first loop 102 of the support element 30m. The reinforcement element 32m then passes through the aperture 98 and then back through the aperture 100, so as to form a second loop 104. The ligament 26 is thus supported by both of the loops 102 and 104, which provides the possibility of a reduction of the width of the reinforcement element 32m in the coupling region 68m, as described above.

Whilst one particular threading arrangement of the reinforcement element 32m through the apertures 94 to 100 is shown in FIG. 16, it will be understood that many different configurations may be employed, depending on factors including the number of loops to be provided in the support element 30m.

Turning now to FIG. 17, there is shown an optional further feature of any one of the support assemblies shown and described in FIGS. 2 to 16. For illustration purposes, the support assembly 24 of FIG. 2 is shown in the drawing.

The drawing is a schematic side (lateral) view of the knee joint 10, showing the location of the mouth 36 of the femoral tunnel portion 18. In the illustrated embodiment, the femoral tunnel portion 18 has emerged from the femoral component 12 in an unplanned position, or other operational limitations (such as physical characteristics of the femoral component 12) have dictated such direction of the tunnel portion. As a result, the mouth 36 of the femoral tunnel portion 18 is in an unplanned or generally undesirable position, in which the resultant direction of loading applied to the fixation device 28 by the reinforcement element 32 would be undesirable. In particular, the loading imparted on the fixation device 28 may be such as to cause rotation of the fixation device, with the possibility of the fixation device being dragged back into the femoral tunnel portion 18.

Accordingly, in this embodiment, a guide member in the form of post 106 is inserted into the femoral component 12. The guide post 106 has a threaded portion (not shown) for inserting the post into the bone, and an unthreaded upper portion. The reinforcement element 32 passes around the post 106, around the unthreaded portion, and from there is directed through the second tibial tunnel 54. The position of the post 106 is selected so that a direction of applied loading on the fixation device 28 is changed, ideally to one which does not result in rotation of the fixation device during use. As a result, the reinforcement element 32 extends from the fixation device 28 to the guide post 106 in a first direction, and then from the guide post 106 to the anchor point in a second, different direction (which is non-parallel to the first direction). Whilst a guide member in the form of the guide post 106 is shown, it will be understood that alternative guide members may be employed, such as a staple. Typically the staple would clamp the reinforcement element 32 to the bone surface, but in a variation the staple may not clamp the reinforcement element, or may carry a post or the like around which the reinforcement element may pass.

Reference is made herein to an extra-articular reinforcement element. It will be understood that this should be taken to mean a reinforcement element which is not situated within the joint in question, and so which is not in an intra-articular position within the joint. In the specific context of the invention, employing a fixation device which is externally located (overlying a mouth of a bone tunnel), the reinforcement element is located outside of the bone tunnel, but it will be understood that the invention should not necessarily be restricted to such.

Various modifications may be made to the foregoing without departing from the spirit or scope of the present invention. For example, any one of the above described embodiments may comprise one or more feature derived from one or more of the other disclosed embodiments. Further embodiments of the invention may comprise features selected from any one of the above described embodiments.

1. A support assembly for positioning a replacement ligament in a bone tunnel formed in a joint between two adjacent bones and reinforcing the joint, the support assembly comprising:
a fixation device for the replacement ligament, the fixation device adapted to overlie a mouth of the bone tunnel and having a bone facing surface which can abut an external surface of the bone to thereby position the ligament in the bone tunnel and support tensile loading on the ligament;
a support element for the replacement ligament, the support element being coupled to the fixation device and adapted to receive the ligament so that the ligament can be secured to the fixation device and thus positioned within the bone tunnel when the fixation device is located so that it overlies the tunnel mouth; and
an extra-articular reinforcement element, the extra-articular reinforcement element being coupled to the fixation device and adapted to extend from the fixation device to an anchor point which is remote from the mouth of the bone tunnel, to provide extra-articular reinforcement for the joint.

2. A support assembly as claimed in claim 1, in which the reinforcement element is coupled to the fixation device by the support element.

3. A support assembly as claimed in claim 1, in which the support element is a first support element, and in which the assembly comprises a second support element for the reinforcement element, for coupling the reinforcement element to the fixation device.

4. A support assembly as claimed in claim 3, in which the first and second support elements extend through a common pair of apertures in the fixation device.

5. A support assembly as claimed in claim 3, in which the fixation device comprises a pair of apertures for the first support element, and a separate pair of apertures for the second support element.

6. A support assembly as claimed in claim 1, in which the reinforcement element is directly coupled to the fixation device, passing through at least one aperture of the fixation device.

7. A support assembly as claimed in claim 6, in which the fixation device comprises a pair of spaced apertures for the reinforcement element, the reinforcement element passing through the pair of apertures so that it is secured to the fixation device.

8. A support assembly as claimed in claim 1, in which the reinforcement element forms a pulling element for pulling the support assembly and a trailing ligament coupled to the fixation device along the bone tunnel.

9. A support assembly as claimed in claim 1, in which:
the fixation device serves for guiding the ligament along the bone tunnel, and is a generally elongate device capable of being manipulated between a pulling position in which it has a reduced lateral extent relative to a pulling direction, to an anchoring position in which it has a maximum lateral extent relative to the pulling direction;
and in which the reinforcement element forms a manipulating element for manipulating the fixation device from the pulling position to the anchoring position.

10. A support assembly as claimed in claim 1, in which the reinforcement element also defines the support element.

11. A support assembly as claimed in claim 10, in which the reinforcement element passes through a first reinforcing aperture in the fixation device in a first direction and then through a second reinforcing aperture in a second direction, to thereby form a loop defining an eye which can receive the replacement ligament.

12. A support assembly as claimed in claim 1, in which the reinforcement element is a woven elongate tape.

13. A support assembly as claimed in claim 1, in which the reinforcement element, when coupled to the fixation device, is folded so that it comprises first and second legs, and in which the reinforcement element is coupled to the fixation device by securing the first and second legs together.

14. A support assembly as claimed in claim 13, in which the reinforcement element is at least partly tubular, at least one aperture provided in a wall of one of the first and second legs so that the other one of the first and second legs may pass into the aperture and along the inside of the other leg, for securing the legs together.

15. A support assembly as claimed in claim 1, in which the reinforcement element comprises a coupling region part way along a length of the element between opposed first and second ends of the element, the coupling region adapted for coupling the reinforcement element to the fixation device.

16. A support assembly as claimed in claim 15, in which the coupling region has a width which is less than a corresponding width of a remainder of the element.

17. A support assembly as claimed in claim 16, in which reinforcement element is woven, comprising a plurality of warps and a plurality of wefts, and in which the coupling region is free from wefts.

18. A support assembly as claimed in claim 10, in which the reinforcement element is coupled to the fixation device in such a way that the reinforcement element defines a plurality of loops which together form the support element.

19. A support assembly as claimed in claim 18, in which the reinforcement element is coupled to the fixation device in such a way that the lengths of the loops are adjustable.

20. A support assembly as claimed in claim 1, comprising a guide member for the reinforcement element, the guide member being implantable in a bone of the joint and shaped to cooperate with the reinforcement element, so as to provide control of a direction of loading applied to the fixation device by the reinforcement element during use.

21. A support assembly as claimed in claim 20, in which the guide member defines a surface around which the reinforcement element can pass so that, in use, the reinforcement element extends from the fixation device, around the guide pin and then to the anchor point.

22. A method of locating a replacement ligament in a bone tunnel formed in a joint between two adjacent bones and of reinforcing the joint, the method comprising the steps of:
securing a replacement ligament to a fixation device of a support assembly using a support element coupled to the fixation device;
inserting the ligament into the bone tunnel;
locating the fixation device so that it overlies a mouth of the bone tunnel with a bone facing surface of the fixation device abutting an external surface of the bone, to thereby position the ligament secured to the fixation device within the bone tunnel and to support tensile loading on the ligament;
extending an extra-articular reinforcement element coupled to the fixation device from the fixation device to an anchor point which is remote from the mouth of the bone tunnel; and
anchoring the reinforcement element at the anchor point, to provide extra-articular reinforcement for the joint.

23. A method as claimed in claim 22, comprising the further steps of:
implanting a guide member for the reinforcement element in a bone of the joint; and
arranging the reinforcement element so that it cooperates with the guide member, so that the guide member provides control of a direction of loading applied to the fixation device by the reinforcement element during use.

24. A method as claimed in claim 23, in which the step of arranging the reinforcement element so that it cooperates with the guide member comprises passing the reinforcement element around a surface of the guide member.

25. A method as claimed in claim 23, comprising locating the reinforcement element so that it extends from the fixation device, around the guide member and then to the anchor point.

26. A method as claimed in claim 23, comprising locating the reinforcement element so that it extends from the fixation device to the guide member in a first direction, and then from the guide member to the anchor point in a second, different direction.
